(19) United States
(12) Patent Application Publication
(10) Pub. No.: US 2002/0137733 A1
(43) Pub. Date: Sep. 26, 2002

(54) Macrocyclic anti-viral compounds

(76) Inventors: Guy Falardeau, Laval (CA); Laval Chan Chun Kong, Kirkland (CA); Jean Bedard, Laval (CA)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/025,987

(22) Filed: Dec. 26, 2001

Related U.S. Application Data
(60) Provisional application No. 60/258,007, filed on Dec. 27, 2000.

(51) Int. Cl. A61K 31/4745; C07D 487/14

(52) U.S. Cl. 514/183; 514/291; 540/456

(57) ABSTRACT

The present invention relates to heterocyclic compounds having antiviral activity. In particular, compounds of

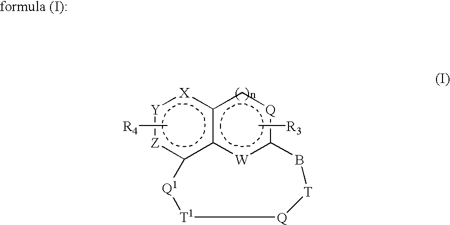

wherein B, W, X, Y, Z, R₂, R₃, R₄, R₅, T, T¹, Q, Q¹ and n are as defined herein, and are useful in the therapy and prophylaxis of viral infection in mammals.

### FIELD OF THE INVENTION

The present invention relates to heterocyclic compounds, and more particularly, to macrocyclic compounds and their use in therapy and prophylaxis of viral infection.

### BACKGROUND OF THE INVENTION

Of the DNA viruses, the herpes group is the source of the most common viral illnesses in man. The group consists of herpes simplex virus (HSV) type I and II, varicella zoster (VZV), Epstein-Barr virus (EBV) and cytomegalovirus (CMV).

As with other herpes viruses, infection with CMV leads to a lifelong association of virus and host. Following a primary infection, virus may be shed for a number of years. Infection in otherwise healthy individuals is frequently asymptomatic, as 80% of the adult population harbor the virus in latent form. In immunocompromised individuals, such as chemotherapy patients, organ transplant patients and in particular AIDS sufferers, latent CMV can be re-activated resulting in microcephaly, hepatosplenomegaly, jaundice, convulsive seizures which may cause mental retardation, mononucleosis, retinitis and even death. In AIDS patients, CMV is a predominant cause of morbidity.

A variety of drugs have been developed to treat herpesvirus infection, including naturally occurring proteins and synthetic nucleoside analogs. For example, the natural antiviral protein, interferon, has been used in the treatment of herpesvirus infections, as have the nucleoside analogs, cytosine-arabinoside, adenine-arabinoside, iodoxyuridine and acyclovir, which is presently the treatment of choice for herpes simplex type I infection.

Unfortunately, drugs such as acyclovir that have proven effective to treat certain herpesviruses infections are not sufficiently effective to treat CMV. And, drugs currently used to treat CMV infection, such as ganciclovir (9-[(1,3-dihyroxy-2-propoxy)methyl]guanine), cidofovir and foscarnet (phosphonoformic acid), lack the acceptable side effect and safety profiles of the drugs approved for treatment of other herpesviruses.

In the case of the treatments for AIDS , combination anti-HIV therapy is now the standard of care for people with HIV. There are now 14 anti-HIV drugs available by prescription. These anti-HIV drugs fall into three categories: nucleosides analogs, which include AZT, ddI, ddC, d4T, abacavir and 3TC™ protease inhibitors which include amprenavir, indinavir, nelfinavir, saquinavir and ritonavir and non-nucleoside reverse transcriptase inhibitors (NNRTI) which include nevirapine, efavirenz and delavirdine. Compared to HIV, there are presently (at least) two licensed therapies for chronic hepatitis B virus infection which is interferon and lamivudine. Other drugs are currently under clinical trials including lamivudine, famciclovir, lobucavir and adefovir. But many studies have shown that most patients relapse after completion of therapy and develop resistance to the drugs.

Development of resistance has recently become a major concern in the treatment of HIV and HBV infections. Resistance usually occurs when the drugs being used are not potent enough to completely stop virus replication. If the virus can reproduce at all in the presence of drugs, it has the opportunity to make changes in its structure, called mutations, until it finds one that allows it to reproduce it spite of the drugs. Once a mutation occurs, it then grows unchecked and soon is the dominant strain of the virus in the individual. The drug becomes progressively weaker against the new strain. There is also increasing concern about cross-resistance. Cross-resistance occurs when mutations causing resistance to one drug also cause resistance to another. Several studies have proven that combining two drugs delays the development of resistance to one or both drugs compared to when either drug is used alone. Other studies suggest that three-drug combinations extend this benefit even further. As a result, many people believe that the best way of preventing, or at least delaying resistance is to use multi-drug combination therapies.

Rhinoviruses are the main etiologic agents of infectious common colds, which represent about 40% of the acute respiratory infections in man. The antigenic diversity of rhinoviruses precludes any prevention by vaccination. In recent years, efforts have concentrated on chemoprophylaxis or chemotherapy with antiviral agents.

Thus, there remains a need for therapeutic and prophylactic non-nucleoside agents effective to treat viral infection.

### SUMMARY OF THE INVENTION

The present invention provides a method of inhibiting viral replication selected from the group consisting of cytomegalovirus (CMV), herpes simplex virus (HSV), influenza, HIV, rhinovirus (RV), Epstein-Barr virus (EBV) and varicella zoster virus (VZV) in a mammal comprising administering to said mammal an anti-viral amount of a compound of formula (I):

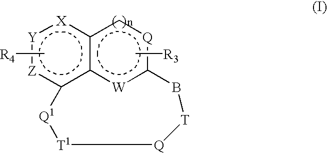

wherein

W is selected from CH, CR₃, CH₂, C═O, CHR₃, N and NR₅; one of X, Y, and Z is N or NR₅ while the other two are

independently selected from CH, CR₄, CH₂, C═O and CHR₄;

B is selected from the group consisting of:

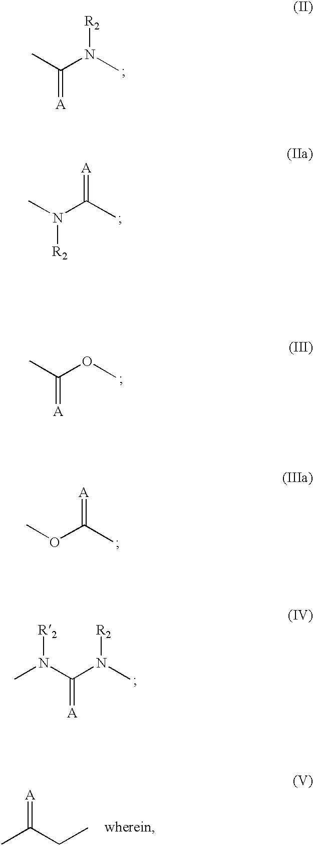

A is O or S;

T and T¹ are independently selected from C₁₋₆ (alkyl, alkoxy, acyl, acyloxy or alkoxycarbonyl), C₂₋₆ alkenyl, C₂₋₆ alkynyl optionally substituted with OH, halogen, amino, mercapto, carboxy or a saturated or unsaturated C₃₋₁₀ (carbocycle or heterocycle) optionally substituted with OH, halogen, amino, mercapto, carboxy, C₁₋₄ (alkyl, alkoxy, alkylthio, acyl, acyloxy or alkoxycarbonyl);

Q and Q¹ are independently selected from N, NR₅, O, S, NH, CH, CHR₃ or a bond;

R₂ and R′₂ are independently selected from H or C₁₋₄ alkyl;

R₃ and R₄ are independently selected from H, OH, halogen, amino, cyano, C₁₋₆ (alkyl, alkoxy, acyl, acyloxy or alkoxycarbonyl), C₂₋₆ alkenyl, C₂₋₆ alkynyl optionally substituted with OH, halogen, amino or C₁₋₄ alkoxy, and saturated or unsaturated C₃₋₁₀ (carbocycle or heterocycle) optionally substituted with OH, halogen, amino, mercapto, C₁₋₄ alkylthio, C₁₋₄ alkoxycarbonyl, halo-substituted C₁₋₄ alkyl or halo-substituted C₁₋₄ alkoxy, C₁₋₄ alkyl, C₁₋₄ alkoxy or C₁₋₄ carboxy;

R₅ is H, C₁₋₆ alkyl or C₁₋₆ acyl optionally substituted with OH, halogen, amino or C₁₋₄ alkoxy; and

n is 0, 1, 2 or 3.

In another embodiment, there is provided viral replication inhibiting compounds and pharmaceutically acceptable salts thereof according to formula (I) for treating or preventing a viral infection selected from the group consisting of cytomegalovirus (CMV), herpes simplex virus (HSV), influenza, HIV, rhinovirus, Epstein-Barr virus (EBV) and varicella zoster virus (VZV).

In another embodiment, there is provided a method of inhibiting viral replication selected from the group consisting of cytomegalovirus (CMV), herpes simplex virus (HSV), influenza, HIV, rhinovirus, Epstein-Barr virus (EBV) and varicella zoster virus (VZV) in a mammal comprising administering to said mammal an anti-viral amount of a compound of formula (I) and at least one further antiviral agent.

In another embodiment, there is provided a pharmaceutical composition for treating or preventing viral infection selected from the group consisting of cytomegalovirus (CMV), herpes simplex virus (HSV), influenza, HIV, rhinovirus, Epstein-Barr virus (EBV) and varicella zoster virus (VZV) comprising at least one compound according to formula (I) together with at least one pharmaceutically acceptable carrier or excipient.

In another embodiment, there is provided a pharmaceutical composition for treating or preventing viral infection selected from the group consisting of cytomegalovirus (CMV), herpes simplex virus (HSV), influenza, HIV, rhinovirus, Epstein-Barr virus (EBV) and varicella zoster virus (VZV) comprising at least one compound according to formula (I) and at least one further antiviral agent.

In another embodiment of the invention is the use of a compound according to formula (I) for the manufacture of a medicament for treating or preventing viral infection selected from the group consisting of cytomegalovirus (CMV), herpes simplex virus (HSV), influenza, HIV, rhinovirus, Epstein-Barr virus (EBV) and varicella zoster virus (VZV) in a host.

### DETAILED DESCRIPTION OF THE INVENTION

In one embodiment, compounds of the present invention comprise those wherein the following embodiments are present, either independently or in combination.

The present invention provides a method of inhibiting viral replication selected from the group consisting of cytomegalovirus (CMV), herpes simplex virus (HSV), influenza, HIV, rhinovirus, Epstein-Barr virus (EBV) and varicella zoster virus (VZV) in a mammal comprising administering to said mammal an anti-viral amount of a compound of formula (I):

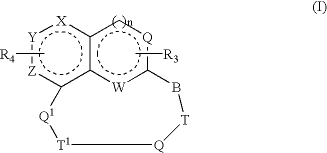

wherein W, X, Y, Z, A, B, Q, Q¹, T, T¹, R₂ to R₅ and n are as defined above.

In one embodiment of the invention, there is provided a method of inhibiting viral replication selected from the group consisting of cytomegalovirus (CMV), herpes simplex virus (HSV), influenza, HIV, rhinovirus, Epstein-Barr virus (EBV) and varicella zoster virus (VZV) in a mammal comprising administering to said mammal an anti-viral amount of a compound of formula (VI):

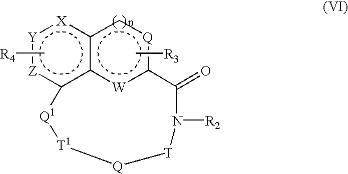

wherein W, X, Y, Z, Q, Q¹, T, T¹, R₂ to R₅ and n are as defined above.

In one embodiment of the invention, there is provided a method of inhibiting viral replication selected from the group consisting of cytomegalovirus (CMV), herpes simplex virus (HSV), influenza, HIV, rhinovirus, Epstein-Barr virus (EBV) and varicella zoster virus (VZV) in a mammal comprising administering to said mammal an anti-viral amount of a compound of formula (VII):

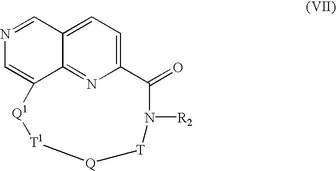

wherein Q, Q¹, T, T¹, R₂ and R₅ are as defined above.

In another embodiment of the present invention, there is provided viral inhibiting compounds and pharmaceutically acceptable salts thereof according to compounds of formula (I), (VI) and (VII) as shown above.

In another embodiment of the present invention, there is provided viral inhibiting compositions comprising a pharmaceutically acceptable carrier, diluent or adjuvant and a compound of formula (I), (VI) and (VII) as shown above or a pharmaceutically acceptable salt thereof.

By the term pharmaceutically acceptable salts of the compounds of formula (I), (VI) and (VII) are meant those derived from pharmaceutically acceptable inorganic and organic acids and bases. Examples of suitable acids include hydrochloric, hydrobromic, sulphuric, nitric, perchloric, fumaric, maleic, phosphoric, glycollic, lactic, salicylic, succinic, toluene-p-sulphonic, tartaric, acetic, citric, methanesulphonic, formic, benzoic, malonic, naphthalene-2-sulphonic and benzenesulphonic acids.

Salts derived from appropriate bases include alkali metal (e.g. sodium), alkaline earth metal (e.g. magnesium), ammonium and NR₄+ (where R is C₁₋₄ alkyl) salts.

References hereinafter to a compound according to the invention includes compounds of the general formula (I) and their pharmaceutically acceptable salts.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control.

In addition, the materials, methods, and examples are illustrative only and not intended to be limiting. As used in this application, the term “alkyl” represents an unsubstituted or substituted (by a halogen, nitro, SO₃R₄, PO₃R₄R₄, CONH₂, COOH, O—C₂₋₆ alkyl, O—C₂₋₆ alkenyl, O—C₂₋₆ alkynyl, C₆₋₁₂ aryl, C₃₋₁₀ heterocycle, hydroxyl, amino, NR₄R₄, or COOQ, wherein Q is C₁₋₆ alkyl; C₂₋₆ alkenyl; C₂₋₆ alkynyl, C₆₋₁₂ aryl and R₄ is H, C₁₋₆ alkyl) straight chain, branched chain or cyclic hydrocarbon moiety (e.g. isopropyl, ethyl, fluorohexyl or cyclopropyl). The term alkyl is also meant to include alkyls in which one or more hydrogen atoms is replaced by an oxygen, (e.g. a benzoyl) or an halogen, more preferably , the halogen is fluoro (e.g. CF₃— or CF₃CH₂—) Similarly the terms “alkenyl” and “alkynyl represent an alkyl containing at least one unsaturated group (e.g. allyl, acetylene, ethylene). For convenience however, the terms “alkoxy”, “alkylthio”, “acyl”, “acyloxy” and “alkoxycarbonyl” refer to chains that are either saturated or unsaturated and may also be straight or branched. Where indicated, any of the above mentioned chains may have various substituents and it is understood that there may be one or more substituents unless otherwise specified.

The term “carbocycle” refers to a cyclic carbon chain or ring which is saturated or unsaturated. A “heterocycle” is a ring incorporating heteroatoms selected from N, O and S in place of carbon. Unsaturated carbocycles and heterocycles may be aromatic i.e. aryl such as phenyl or naphthyl, or heteroaryl such as pyridine, quinoline, epoxide; furan; benzofuran; isobenzofuran; oxathiolane; dithiolane; dioxolane; pyrrole; pyrrolidine; imidazole; pyrimidine; indole; piperidine; morpholine; thiophene and thiomorpholine. Where indicated, any of the above mentioned rings may have various substitutions. It is understood that there may be one or more substituents unless otherwise specified.

The term “amino” includes primary amines i.e. NH₂, secondary amines i.e. NHR, or tertiary amines i.e. N(R)₂ wherein R is C₁₋₄ alkyl. Also encompassed by the term are quaternary amines such as NH₃⁺. When there is a sulfur atom present, the sulfur atom can be at different oxidation levels, ie. S, SO, or SO₂. All such oxidation levels are within the scope of the present invention. In methods of the present invention, viral replication is inhibited by administering compounds of formula (I) as shown above, wherein:

W is selected from CH, CR₃, CH₂, C═O, CHR₃, N and NR₅; and one of X, Y, and Z is N or NR₅ while the other two are independently selected from CH, CR₄, CH₂, C═O and CHR₄. It will be appreciated that the macrocyclic compounds of the invention may be saturated, unsaturated or partially unsaturated and that W, X, Y and Z will have the appropriate valency for each condition. For example, when the rings are unsaturated, W may be N, CH or CR₃. And conversely, when the rings are saturated W may be CH₂, C═O, CHR₃, NH or NR₅. The same principle applies for X, Y and Z.

In another embodiment n is 0.

In another embodiment W is N or NR₅. In another embodiment Y is N or NR₅, while X and Z are independently CH, CR₄, CH₂, C═O or CHR₄. In another embodiment the heterobicyclic ring incorporating W, X, Y and Z is unsaturated.

In another embodiment, W and Y are independently N or NR₅ while X and Z are independently CH, CR₄, CH₂, C═O or CHR₄. In another embodiment, W and Y are both N while X and Z are CH or CR₄ and the heterobicyclic ring is unsaturated. In another embodiment, W and Y are both N while X and Z are CH or CR₄, the heterobicyclic ring is unsaturated and n is 1, thereby forming a 1,6-naphthyridine ring.

In another embodiment, B is

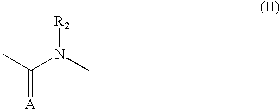

In another embodiment, B is as above and A is O.

In one embodiment, T is chosen from C₁₋₆ (alkyl, alkoxy, acyl, acyloxy or alkoxycarbonyl), C₂₋₆ alkenyl, C₂₋₆ alkynyl optionally substituted with OH, halogen, amino, mercapto, carboxy or a saturated or unsaturated C₃₋₁₀ (carbocycle or heterocycle).

In another embodiment, T is C₁₋₆ alkyl optionally substituted with a saturated or unsaturated C₃₋₁₀ (carbocycle or heterocycle).

In still another embodiment, T is C₁₋₆ alkyl optionally substituted with phenyl.

In another embodiment, T is methyl optionally substituted with a phenyl.

In one embodiment, T¹ is chosen from C₁₋₆ (alkyl, alkoxy, acyl, acyloxy or alkoxycarbonyl), C₂₋₆ alkenyl, C₂₋₆ alkynyl optionally substituted with OH, halogen, amino, mercapto, carboxy or a saturated or unsaturated C₃₋₁₀ (carbocycle or heterocycle.

In another embodiment, T¹ is C₁₋₆ alkyl optionally substituted with a saturated or unsaturated C₃₋₁₀ (carbocycle or heterocycle).

In another embodiment, T¹ is C₁₋₆ alkyl optionally substituted with phenyl.

In another embodiment, T¹ is methyl optionally substituted with phenyl.

In still another embodiment, T¹ is C₂₋₆ alkenyl.

In still another embodiment, T¹ is vinyl.

In still another embodiment, T¹ is allyl.

In one embodiment, Q is chosen from N, O, S.

In another embodiment, Q is O.

In another embodiment, Q¹ is a bond.

In another embodiment, R₂ and R′₂ are H.

In another embodiment, R₃ and R₄ are H, OH, halogen, amino, cyano, C₁₋₆ (alkyl, alkoxy, acyl, acyloxy or alkoxycarbonyl), C₂₋₆ alkenyl, C₂₋₆ alkynyl.

In another embodiment, R₃ and R₄ are H, OH, halogen, amino, cyano, C₁₋₆ (alkyl).

In another embodiment, R₃ and R₄ are H.

In another embodiment, R₅ is H.

In one embodiment, a compound of formula (I) includes the following macrocycle compound:

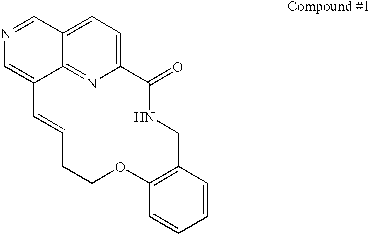

In another embodiment, a compound of formula (I) includes the following macrocycle compound:

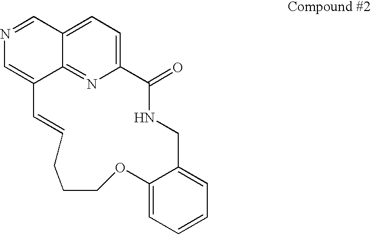

In another embodiment, a compound of formula (I) includes the following macrocycle compound:

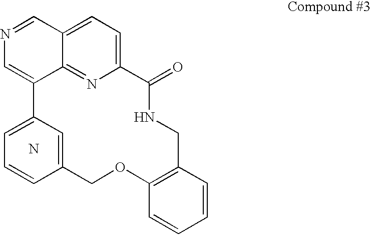

According to methods of the present invention, compounds of formula(I) are administered to a mammal to inhibit replication of or reduce cytopathic effects of viruses. In particular the HIV virus which is known to be the causative agent in Acquired Immune Deficiency Syndrome (AIDS). Other viruses inhibited with compounds of formula(I) include but are not limited to cytomegalovirus (CMV), HSV-1 (herpes simplex virus type 1), HSV-2 (herpes simplex virus type 2), HBV hepatitis B virus), HCV (hepatitis C virus), HPV (human papilloma virus), influenza A, Influenza B, RSV (respiratory syncitial virus), RV (rhinovirus), AV (adenovirus), PIV, Epstein-Barr virus (EBV) and varicella zoster virus (VZV). Furthermore, compounds of formula (I) interact with the nuclear factor k B (NFkB) signal transduction pathway. Consequently compounds of formula (I) may be used to treat conditions mediated by tumour necrosis factor (TNFa) or other cytokines under transcriptional control of NFkB. Conditions include acute and chronic inflammatory diseases such as rheumatoid arthritis, osteoarthritis, Krohn's disease, colitis, and septic shock.

In accordance with the present there is provided compounds characterized by a macrocyclic moiety as illustrated in formula (I) which inhibit viral replication selected from the group consisting of cytomegalovirus (CMV), herpes simplex virus (HSV), influenza, HIV, rhinovirus (RV), Epstein-Barr virus (EBV) and varicella zoster virus (VZV) in a mammal.

In another embodiment, the present invention provides compounds characterized by a macrocyclic moiety as illustrated in formula (I) which inhibit cytomegalovirus (CMV) replication.

In another embodiment, the present invention provides compounds characterized by a macrocyclic moiety as illustrated in formula (I) which inhibit herpes simplex virus (HSV) replication.

In another embodiment, the present invention provides compounds characterized by a macrocyclic moiety as illustrated in formula (I) which inhibit influenza replication.

In another embodiment, the present invention provides compounds characterized by a macrocyclic moiety as illustrated in formula (I) which inhibit HIV replication.

In another embodiment, the present invention provides compounds characterized by a macrocyclic moiety as illustrated in formula (I) which inhibit rhinovirus (RV) replication.

In another embodiment, the present invention provides compounds characterized by a macrocyclic moiety as illustrated in formula (I) which inhibit Epstein-Barr virus (EBV).

In another embodiment, the present invention provides compounds characterized by a macrocyclic moiety as illustrated in formula (I) which inhibit varicella zoster virus (VZV).

Compounds of the present invention can be synthesized using conventional preparative steps and recovery methods known to those skilled in the art of organic chemistry. A preferred synthetic route for producing compounds of formula (I) involves coupling a carboxylic acid intermediate with an amino intermediate. The reaction will be under suitable conditions for amide bond formation i.e. in the presence of a suitable coupling agent such as EDCl or dCC, to yield intermediate compound. The general reaction is illustrated in scheme 1, below:

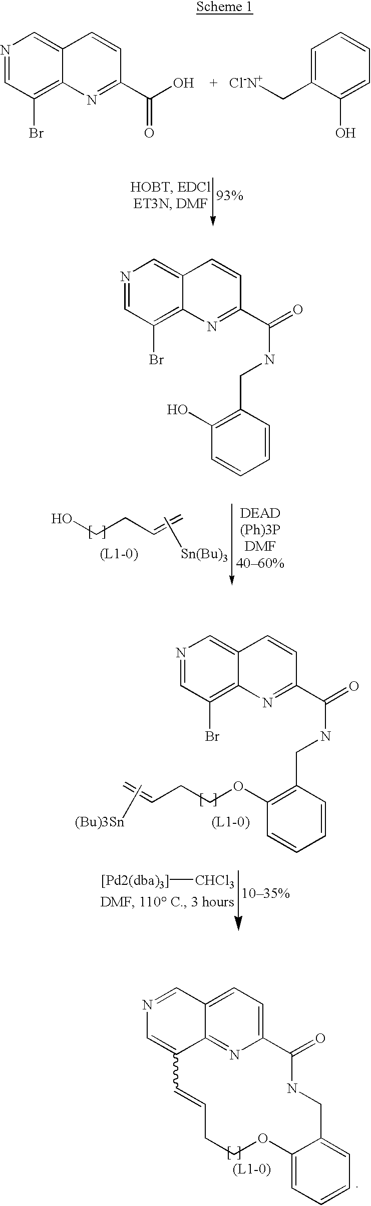

In this general scheme, the stannane is vinyl, but could also be an aryl stannane.

It will be appreciated by those skilled in the art that the compounds of formula I depending on the substituents may contain one or more chiral centers and thus exist in the form of many different isomers, optical isomers (i.e. enantiomers) and mixtures thereof including racemic mixtures. All such isomers, enantiomers and mixtures thereof including racemic mixtures are included within the scope of the present invention.

The present invention also provides anti-viral compositions which comprise a pharmaceutically acceptable carrier or adjuvant and an amount of a compound of formula I effective to inhibit viral replication in a mammal. The proportion of each carrier, diluent or adjuvant is determined by the solubility and chemical nature of the compound and the route of administration according to standard pharmaceutical practice.

Therapeutic and prophylactic methods of this invention comprise the step of treating patients in a pharmaceutically acceptable manner with those compounds or compositions. Such compositions may be in the form of tablets, capsules, caplets, powders, granules, lozenges, suppositories, reconstitutable powders, or liquid preparations, such as oral or sterile parenteral solutions or suspensions. Compounds of the invention may also be administered via an intraocular implant for treating retinitis as a result of CMV infection. In particular, compounds may be embedded in a polymer based implant which will be release into the eye over an extended period of time.

In order to obtain consistency of administration, it is preferred that a composition of the invention is in the form of a unit dose. The unit dose presentation forms for oral administration may be tablets and capsules and may contain conventional excipients. For example, binding agents, such as acacia, gelatin, sorbitol, or polyvinylpyrrolidone; fillers, such as lactose, sugar, maize-starch, calcium phosphate, sorbitol or glycine; tableting lubricants such as magnesium stearate; disintegrants, such as starch, polyvinylpyrrolidone, sodium starch glycollate or microcrystalline cellulose; or pharmaceutically acceptable wetting agents such as sodium lauryl sulphate.

The compounds may be injected parenterally; this being intramuscularly, intravenously, or subcutaneously. For parenteral administration, the compound may be used in the form of sterile solutions containing other solutes, for example, sufficient saline or glucose to make the solution isotonic. The amount of active ingredient administered parenterally will be approximately 0.01 to 250 mg/kg/day, preferably about 1 to 10 mg/kg/day, more preferably about 0.5 to 30 mg/kg/day, and more most preferably about 1-20 mg/kg/day.

The compounds may be administered orally in the form of tablets, capsules, or granules containing suitable excipients such as starch, lactose, white sugar and the like. The compounds may be administered orally in the form of solutions which may contain coloring and/or flavoring agents. The compounds may also be administered sublingually in the form of tracheas or lozenges in which each active ingredient is mixed with sugar or corn syrups, flavoring agents and dyes, and then dehydrated sufficiently to make the mixture suitable for pressing into solid form. The amount of active ingredient administered orally will depend on bioavailability of the specific compound.

The solid oral compositions may be prepared by conventional methods of blending, filling, tableting, or the like. Repeated blending operations may be used to distribute the active agent throughout those compositions employing large quantities of fillers. Such operations are, of course, conventional in the art. The tablets may be coated according to methods well known in normal pharmaceutical practice, in particular with an enteric coating.

Oral liquid preparations may be in the form of emulsions, syrups, or elixirs, or may be presented as a dry product for reconstitution with water or other suitable vehicle before use. Such liquid preparations may or may not contain conventional additives. For example suspending agents, such as sorbitol, syrup, methyl cellulose, gelatin, hydroxyethylcellulose, carboxymethylcellulose, aluminum stearate gel, or hydrogenated edible fats; emulsifying agents, such as sorbitan monooleate or acaci; non-aqueous vehicles (which may include edible oils), such as almond oil, fractionated coconut oil, oily esters selected from the group consisting of glycerine, propylene glycol, ethylene glycol, and ethyl alcohol; preservatives, for instance methyl para-hydroxybenzoate, ethyl para-hydroxybenzoate, n-propyl parahydroxybenzoate, or n-butyl parahydroxybenzoate of sorbic acid; and, if desired, conventional flavoring or coloring agents.

For parenteral administration, fluid unit dosage forms may be prepared by utilizing the peptide and a sterile vehicle, and, depending on the concentration employed, may be either suspended or dissolved in the vehicle. Once in solution, the compound may be injected and filter sterilized before filling a suitable vial or ampoule and subsequently sealing the carrier or storage package. Adjuvants, such as a local anesthetic, a preservative or a buffering agent, may be dissolved in the vehicle prior to use. Stability of the pharmaceutical composition may be enhanced by freezing the composition after filling the vial and removing the water under vacuum, (e.g., freeze drying the composition). Parenteral suspensions may be prepared in substantially the same manner, except that the peptide should be suspended in the vehicle rather than being dissolved, and, further, sterilization is not achievable by filtration. The compound may be sterilized, however, by exposing it to ethylene oxide before suspending it in the sterile vehicle. A surfactant or wetting solution may be advantageously included in the composition to facilitate uniform distribution of the compound.

The pharmaceutical compositions of this invention comprise an antiviral replication inhibiting amount of a compound of formula I and a pharmaceutically acceptable carrier, diluent or adjuvant. Typically, they contain from about 0.1% to about 99% by weight of active compound, and preferably from about 10% to about 60% by weight depending on which method of administration is employed.

An antiviral replication inhibiting amount is that amount of active compound required to slow the progression of viral replication or reduce viral load from that which would otherwise occur without administration of said compound. Or, it is an amount of active compound required to slow the progression or reduce the intensity of symptoms resulting from viral infection or elimination thereof.

Viral inhibiting activity of compounds of the invention can be determined according to the plaque reduction assay described in detail in the examples. Under these particular conditions, a compound having such activity will exhibit an IC₅₀ of approximately 50 μg/ml or less, preferably 25 μg/ml or less, more preferably 10 μg/ml or less, and most preferably less than 1 μg/ml.

Physicians will determine the dosage of the present therapeutic agents which will be most suitable. Dosages may vary with the mode of administration and the particular compound chosen. In addition, the dosage may vary with the particular patient under treatment. The dosage of the compound used in the treatment will vary, depending on viral load, the weight of the patient, the relative efficacy of the compound and the judgment of the treating physician.

Such therapy may extend for several weeks or months, in an intermittent or uninterrupted manner.

To further assist in understanding the present invention, the following non-limiting examples are provided.

### EXAMPLE 1

Synthesis

Preparation of Compound #1

Step 1

### 8-bromo-[1,6]Naphthyridine-2-carboxylic acid 2-hydroxybenzylamine

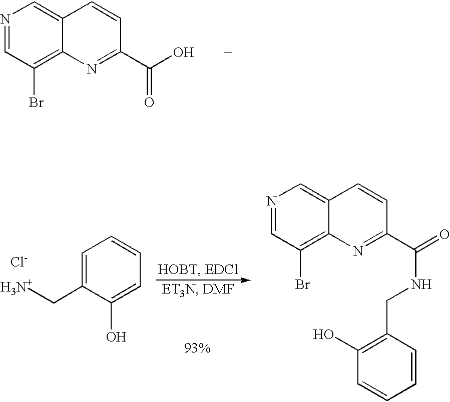

Triethylamine (1.65 mL, 11.8 mmol) was added to a solution of the salt (406 mg, 2.54 mmol) in DMF (4 mL) at room temperature. The solution was stirred at room temperature for five minutes. Simultaneously, the acid (85 mg, 3.39 mmol), HOBT (50 mg, 3.73 mmol) and EDCI (715 mg, 3.73 mmol)were added. The reaction was left to stir overnight at room temperature. The resulting suspension was filtered and the cake was washed with cold methanol and acetone. The mother liquor was evaporated to dryness then suspended in acetone and filtered, the cake was washed with cold methanol. The two solids were combined and dried under vacuum to yield the title compound in a 93% yield.

¹H NMR (400 MHz) (DMSO) δ: 9.79(s, 1H), 9.50 (s, 1H), 9.13 (t, 1H, J=6 Hz), 8.87 (d, 1H, J=8.5 Hz), 7.21 (d, 1H, J=7 Hz), 7.10 (t, 1H, J=7.5 Hz), 6.86 (d, 1H, J=8 Hz), 6.77 (t, 1H, J=7.5 Hz), 4.57 (d, 2H, J=6.5 Hz)

Step 2

### 8-bromo-[1,6]naphthyridine-2-carboxylic acid 2-(4-tributylstannanyl-but-3-enyloxy)-benzylamide

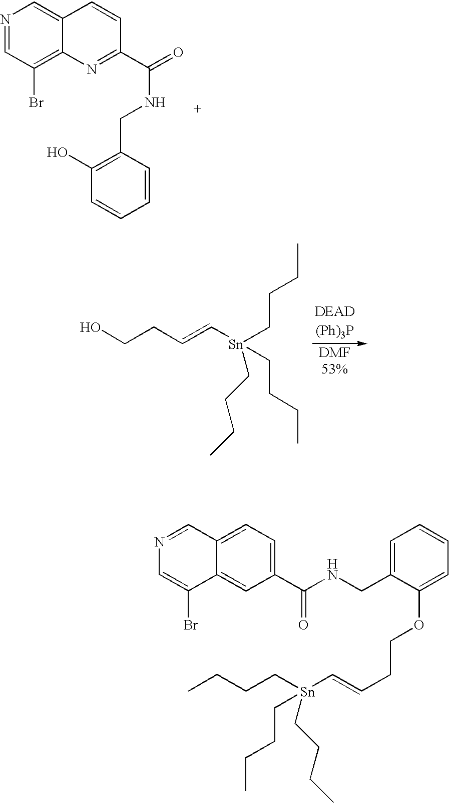

To a solution of the naphthyridine (213.5 mg, 0.59 mmol), the stannane (186.6 mg, 0.54 mmol) and the triphenylphosphine (154.7 mg, 0.59 mmol) in DMF (2 mL) under dry nitrogen at room temperature was added DEAD (0.94 mL, 0.59 mmol) over a period of ten minutes. The solution was stirred over night at room temperature. The solution was evaporated to dryness and the residue was dissolved in a minimum of CH₂Cl₂ and purified using flash chromatography (250 mL of gel, 40% AcOEt/He to yield the title compound in a 53% yield.

¹H NMR (400 mhz) (CDCl₃) δ: 9.25 (s, 1H), 9.03 (s, 1HO, 8.86 (t, 1H, J=6 Hz, NH), 8.52 (d, 1H, J=8.5 Hz), 8.46 (d, 1H, J=8.5 Hz), 7.40 (d, 1H, J=7.5 Hz), 7.30-7.25 (m, 1H), 6.96-6.92 (m, 2H), 6.24-5.93 (m, 2H), 4.75 (d, 2H, J=6.5 Hz), 4.15 (t, 2H, J=7 Hz), 2.78 (q, 2H, J=7 Hz), 1.53-1.42 (m, 6H), 1.35-1.21 (m,6H), 1-0.78 (m, 15H)

Step 3

Macrocycle

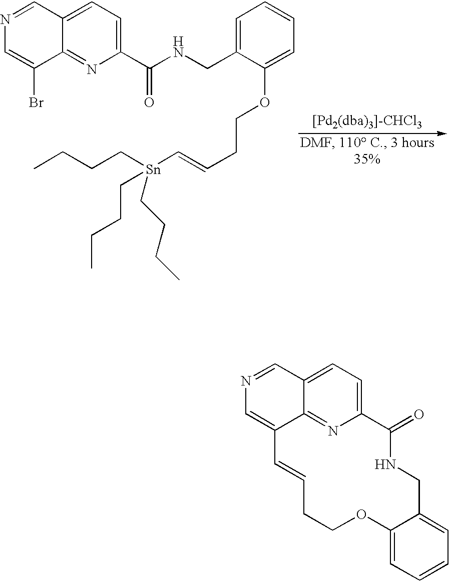

To a solution of the naphthyridine (78 mg, 0.11 mmol) in DMF (1 mL) under dry nitrogen at 110° C. was added Tris(dibenzylideneacetone)-dipalladium(0)-chloroform adduct (11 mg, 0.011 mmol) and stirred at 110° C. After stirring for 2 hours, another portion of Tris(dibenzylideneacetone)-dipalladium(0)-chloroform adduct (11 mg, 0.011 mmol) was added and stirred at 110° C. for an additional hour. The solution was evaporated to dryness and the residue was dissolved in a minimum of CH₂Cl₂ and purified using flash chromatography (40 mL of gel, 30% AcOEt/He to 100% AcOEt). The resulting solid was triturated with pentane several times and the resulting composition was dried under vacuum to yield compound #1 in a 35% yield.

¹H NMR (400 MHz, CDCl₃) δ: 10.03 (bs, 1H), 9.20 (bs, 1H), 8.69 (bs, 1H), 8.47 (d, 1H, J=8.5 Hz), 8.34 (d, 1H, J=8.5 Hz), 7.82 (dt, 1H, J=16.8 Hz), 7.35-7.25 (m, 2H), 7.02-6.93 (m, 2H), 6.73 (d, 1H, J=16 Hz), 4.76 (d, 2H, J=6.5 Hz), 4.40 (t, 2H, J=6 Hz), 2.93 (q, 2H, J=6 Hz)

### EXAMPLE 2

Preparation of Compound #2

Step 1

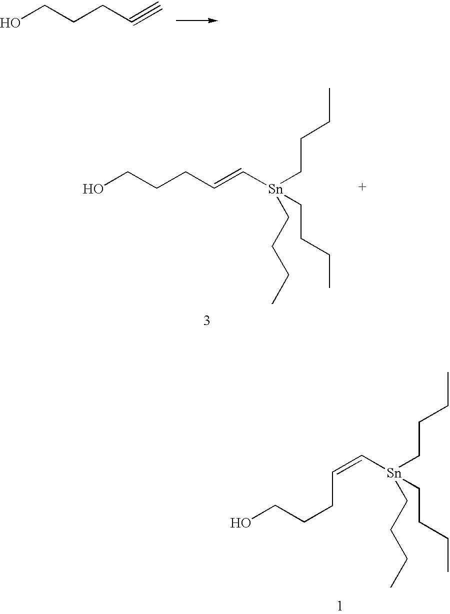

In a dry flask under nitrogen, pentynol (904 mg, 10.7 mmol) and AIBN were charged and stirred at room temperature for 15 minutes and tributyl tin hydride was added and stirred for an additionnal 15 minutes then heated at 120° C. for 2 hrs. The crude reaction was used directly in the next step. Crude yield was quantitative.

¹H NMR (400 MHz) (CDCl₃) trans isomer: 5.99-5.90 (m, 2H), 3.76-3.58 (m, 2H), 2.27-2.22 (m, 2H), 1.75-1.65 (m, 2H), 1.62-1.39 (m, 6H), 1.36-1.22 (m, 6H), 1.16-0.78 (m, 15H)

Step 2

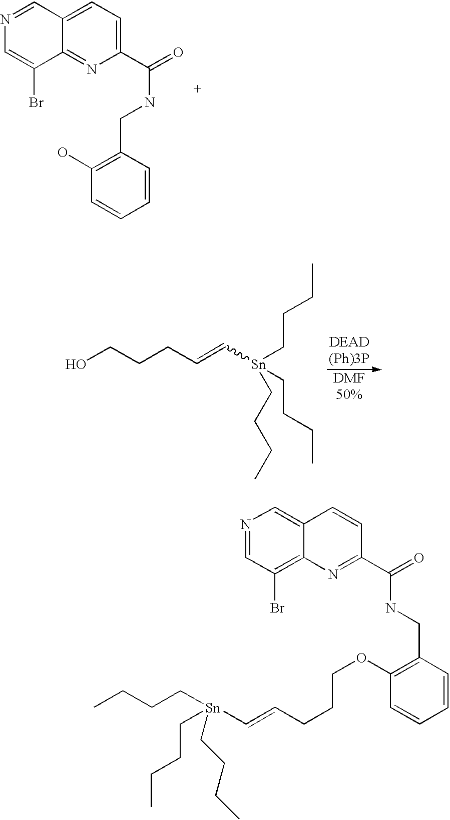

To a solution of the naphthyridine (949 mg, 2.65 mmol) (prepared in a similar manner as in Example 1), the stannane (835 mg, 2.41 mmol) and the triphenylphosphine (695 mg, 2.65 mmol) in DMF (14 mL) under dry nitrogen at room temperature was added DEAD (0.42 mL, 2.65 mmol) over a period of 10 minutes. The solution was stirred at room temperature over night. A precipitate formed and the suspension was filtered. The mixture was diluted with a 1:1 mixture of hexane and ethyl acetate, washed with water and extracted with a 1:1 mixture of hexane and ethyl acetate. (2×). The combined organic phases were dried and purified using a biotage with a 25% EtOAc/He eluant giving the title compound in a 50% yield.

¹H NMR (400 MHz) (CDCl₃): 9.27 (s, 1H), 9.06 (s, 1H), 8.86 (t, 1H, J=6 Hz, NH), 8.54 (d, 1H, J=8.5 Hz), 8.48 (d, 1H, J=8.5 Hz), 7.82 (dd, 1H, J=7.5, 1.5 Hz), 7.31-7.25 (m, 1H), 6.96-6.88 (m, 2H), 6.19-5.85 (m, 2H), 4.76 (d, 2H, J=6.5 Hz), 4.18 (t, 2H, J=7 Hz), 2.78 (q, 2H, J=6.5 Hz), 1.53-1.42 (m, 6H), 1.35-1.21 (m, 6H), 1-0.78 (m, 17H)

Step 3

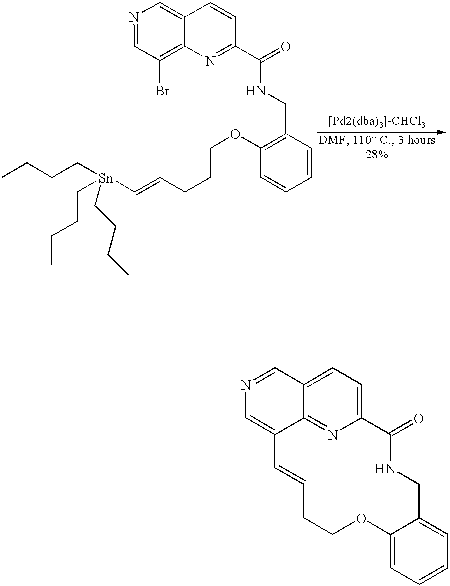

To a solution of the naphthyridine (95 mg, 0.132 mmol) in DMF (2.5 mL) under dry nitrogen at 110° C. was added Tris(dibenzylideneacetone)-dipalladium(0)-chloroform adduct (13 mg, 0.013 mmol) and stirred at 110° C. After stirring for 2 hours, another portion of Tris(dibenzylideneacetone)-dipalladium(0)-chloroform adduct (13 mg, 0.013 mmol) was added and stirred at 110° C. for an additionnal hour. The solution was evaporated to dryness and the residue was purified using flash chromatography (40 mL of gel, 30% AcOEt/He to 100% AcOEt). The resulting solid was triturated in pentane several times and dried under vacuum to yield the compound #2 in a 28% yield.

¹H NMR (400 MHz) (CDCl₃) □: 10.03 (bs, 1H), 9.32 (bs, 1H), 8.60 (m, 2H), 8.50 (d, 1H, J=8 Hz), 7.72 (dt, 1H, J=16, 8 Hz), 7.34 (d, 1H, J=7 Hz), 7.28 (d, 1H, J=7 Hz), 7.97-6.90 (m, 2H), 6.67 (d, 1H, J=15 Hz), 4.69 (d, 2H, J=6.5 Hz), 4.33 (t, 2H, J=5 Hz), 2.7 (m, 2H), 2.26 (m, 2H).

### EXAMPLE 3

Antiviral Assays

The antiviral activity of the compounds for the various viruses was assayed according to the methods described below.

The general procedure for the inhibition of viral cytopathic effect is decribed as follows.

Method 1. Inhibition of Viral Cytopathic Effect (CPE)

This test, run in 96-well flat-bottomed micro plates, is used for the initial antiviral evaluation of all new test Compounds. In this CPE inhibition test, seven one-half log₁₀ dilutions of each test Compound are added to 4 cups containing the cell monolayer; within 5 min., the virus is added and the plate sealed, incubated at 37° C. and CPE read microscopically when untreated infected controls develop a 3 to 4+ CPE (approximately 72 hr to 168 hr depending on the virus). A known positive control drug (ribavirin, HPMPA, acyclovir, ganciclovir, depending on the virus) is evaluated in parallel with test drugs in each test.

The data are expressed as 50% effective (virus-inhibitory) concentrations (EC50).

Method 2. Neutral Red (NR) Dye Uptake

This test is run to validate the CPE inhibition seen in the initial test, and utilizes the same 96-well micro plates after the CPE has been read. Neutral red is added to the medium; cells not damaged by virus take up a greater amount of dye, which is read on a computerized microplate autoreader. An EC50 is determined from this dye uptake.

Method 3. Anti Hiv Activity

The anti-HIV activity of test compounds was evaluated according to standard procedures similar to those described in Ojwang et al (J. Acquired Immune Deficiency Syndromes, 1994,7:560).

Method 4. Anti-HCMV Assay

Human embryonic lung fibroblast cells (HEL) were grown in 96-well plates at the confluent stage and then were infected with reference strains of HCMV Davis at 8, 20, 38 plaque-forming units (PFU)/well for plaque assay or at 100 PFU/well for cytopathic effect (CPE) assay. After a 2 hours incubation, residual virus was removed and the infected cells were further incubated with Eagle's MEM culture medium supplemented with 2% inactivated FCS (fetal calf serum), 1% L-glutamine and 0.3% sodium bicarbonate containing dilution of the test compounds (in duplicate). After 7 days incubation at 37 C. in 5% CO₂ atmosphere, cells were fixed with ethanol and stained with 2.5% Giemsa solution. Virus plaque formation or viral cytopathic effect were monitored microscopically. The antiviral activity is expressed as IC₅₀ which represents the compound concentration required to reduce virus plaque formation or cytopathicity by 50%. IC₅₀ values were estimated from graphic plots of the number of plaques (percentage of control) or percentage of cytopathocity as a function of the concentration of the test compounds. Control compounds ganciclovir (GCV) and cidofovir ([(S)-1-(3-hydroxy-2-phosphonylmethoxypropyl)cytosine], HPMPC) were run in parallel. The results are presented in Table 4.

Method 5. Anti-HSV Assay

Human embryonic lung fibroblast (HEL) cells and Vero cells were propagated in minimal essential medium (MEM) supplemented with 10% fetal calf serum, L-glutamine, and bicarbonate. A CPE assay was used, confluent cultures of HEL or Vero cells grown in 96-well microtiter plates were inoculated with 100 times the 50% cell culture infective dose of the different HSV strains (HSV-1 KOS; HSV-1 Tk−, which is deficient for thymidine kinase; and HSV-2 G). Compounds were added after a 2 hours virus adsorption period. After 2 to 3 days incubation at 37 C. in 5% CO₂ atmosphere, cells were fixed with ethanol and stained with 2.5% Giemsa solution. Virus-induced cytopathic effect (CPE) was then recorded microscopically. The antiviral activity is expressed as IC₅₀ which represents the compound concentration required to reduce cytopathicity by 50%. IC₅₀ values were estimated from graphic plots of the number of plaques (percentage of control) or percentage of cytopathocity as a function of the concentration of the test compounds. Control compounds ganciclovir (GCV) and cidofovir ([(S)-1-(3-hydroxy-2-phosphonylmethoxypropyl)cytosine], HPMPC) were run in parallel. The results are presented in Table 1.

Method 6. Anti-VZV Assay

Human embryonic lung fibroblast (HEL) cells were grown in 96-well plates at the confluent stage and then were infected with reference strains VZV expressing viral thymidine kinase (YS and Oka) or lacking the viral thymidine kinase (07-1 and YS-R) at 20 plaque-forming units (PFU) for plaque assay. After a 2 hours incubation, residual virus was removed and the infected cells were further incubated with Eagle's MEM culture medium supplemented with 2% inactivated FCS (fetal calf serum), 1% L-glutamine and 0.3% sodium bicarbonate containing dilution of the test compounds (in duplicate) After 5 days incubation at 37 C. in 5% CO2 atmosphere, cells were fixed with ethanol and stained with 2.5% Giemsa solution. Virus plaque formation was monitored microscopically. The antiviral activity is expressed as IC₅₀ which represents the compound concentration required to reduce virus plaque formation by 50%. IC₅₀ values were estimated from graphic plots of the number of plaques (percentage of control) as a function of the concentration of the test compounds. Control compounds acyclovir (ACV) and brivudin ([(E)-5-(2-bromovinyl)-2′-deoxyuridine], BVDU) were run in parallel. The results are presented in Table 3.

Method 7. Anti-EBV Assay

To determine the effects of the compounds on EBV replication, exponentially growing P3HR-1 cells were treated for 14 days with various concentrations of the compounds. The cells were then harvested, and the genome copy numbers were determined using EBV-specific DNA/DNA hybridization technique. The 50% effective compound concentration (IC₅₀*) was determined from semilogarithmic plot of drug concentrations against the number of viral genome copies per cell, assuming the residual genome level (30 copies per cell) is achieved by an effective compound concentration as 0% and the viral genome level in the controls with no drug as 100%. Control compound cidofovir ([(S)-1-(3-hydroxy-2-phosphonylmethoxypropyl)cytosine], HPMPC) was run in parallel. The results are presented in Table 2.

### EXAMPLE 4

Methods for Cytotoxicity Assays

Method 8. Neutral Red Uptake

In the neutral red dye uptake phase of the antiviral test described above, the two toxicity control wells also receive neutral red and the degree of color intensity is determined spectrophotometrically. A neutral red CC₅₀ (NRCC₅₀) is subsequently determined.

Data Analysis: Each test Compound's antiviral activity is analysed for the selectivity index (SI), which is the CC50 divided by the EC50.

Special procedures: Except where noted, test Compounds will be solubilized in 100% DMSO at a concentration of 10 mg/ml, then diluted until DMSO is no longer toxic to the cells.

Method 9. Cytotoxicity Measurements Based on the Inhibition of Cell Growth

Cells were seeded at a rate of 5×10³ cells/well in 96-well plates and allowed to proliferate 24 hours. Different concentrations of the test compounds were then added (in duplicates), and after 3 days of incubation at 37 C. in 5% CO₂ atmosphere, the cell number was determined with a coulter counter. Cytotoxicity is expressed as CC₅₀ which represents the compound concentration required to reduce cel growth by 50%.

Method 10. Cytotoxicity Measurements Based on Alteration of Cell Morphology

Minimum toxic concentration which is expressed as MTC, is the minimum concentration of compound required to cause microscopically detectable alteration in normal cell morphology.

Method 11. Cytotoxicity Measurements Based on Reduction of Total Cellular DNA Content Expressed as CC₅₀*

The reduction of total cellular DNA content expressed as CC₅₀* is the concentration required to reduce the total DNA content by 50% using DNA hybridization technique.

TABLE 1

| | ANTI-HSV ACTIVITY EC₅₀ (ug/ml) | | | | | | CYTOTOXICITY | |
|---|---|---|---|---|---|---|---|---|
| | CPE assay | | | | | | (ug/ml) | |
| | HSV-1-KOS | | HSV-2-G | | HSV-1Tk- | | MTC | |
| COMPOUNDS | Vero | HEL | Vero | HEL | Vero | HEL | Vero | HEL |
| Compound #1 | 0,52 | 0,098 | 0,009 | 0,138 | 1,1 | 0,062 | 25 | 6 |
| Compound #2 | 1 | 0,781 | 0,004 | 0,246 | 1,8 | 0,224 | 6 | 100 |
| ACV | 2,4 | ND | 1,3 | ND | 33 | ND | >10 | ND |
| GCV | 2,6 | 0,01 | 1,2 | 0.07,0.024 | >100 | 12.5,17.4 | >100 | >100 |
| HPMPC | 6,7 | 0,098 | 1,3 | 0,01 | 2,4 | 0,012 | >100 | >100 |

TABLE 2

| | ANTI-EBV ACTIVITY EC₅₀ | | |
|---|---|---|---|
| | (ug/ml) | CYTOTOXICITY P3HR cells (ug/ml) | |
| COMPOUNDS | DNA hybridiation assay | CC50 | CC50* |
| Compound #1 | <0.2 | 0,7 | 2,6 |
| HPMPC | <25 | 30 | >25 |

TABLE 3

| | ANTI-VZV ACTIVITY EC₅₀ | | | | |
|---|---|---|---|---|---|
| | (ug/ml) | | | | |
| | Plaque assay | | | | CYTOTOXICITY |
| | VZV Tk+ | | VZV Tk− | | HEL cells |
| | | OKA | 07/1 | YS/R | (ug/ml) |
| COMPOUNDS | YS strain | strain | strain | strain | MTC |
| Compound #1 | 0,05 | 0,06 | 0,06 | 0,05 | >2 |
| ACV | 0,69 | 0,35 | 9 | 12 | >50 |
| BVDU | 0,0013 | 0,0013 | 4 | 39 | >50 |

TABLE 4

| | ANTI-HCMV ACTIVITY EC₅₀ (ug/ml) | | | | | |
|---|---|---|---|---|---|---|
| | HCMV-Davis | | | | | |
| | CPE | | | | CYTOTOXICITY HEL cells | |
| | 100/well | Plaque assay | | | (ug/m/) | |
| COMPOUNDS | (96 well) | 8/well | 20/well | 38/well | MTC | CC50 |
| Compound #1 | 0.003,0.01 | <0.0015 | <0.0015 | 0,003 | 1.6,0.1 | 0,1 |
| Compound #2 | 0.002,0.004 | <0.0015 | 0,003 | 0,004 | 6.3,0.4 | 0,2 |
| GCV | 0.98,0.78 | 0.94,0.86 | 2.7,1.2 | >100 | >100 | 23 |
| HPMPC | 0,1 | 0,15 | 0,14 | 1,9 | >100 | 11 |

The abbreviations used for tables 1 to 4 are as follows

EC₅₀: Concentration required to inhibit viral replication by 50% (CPE or Plaque reduction assays)

EC₅₀*: Concentration required to reduce HBV DNA content by 50% (EBV-specific DNA/DNA hybridization)

CC₅₀: Concentration required to inhibit the exponential growth of u uninfected cells by 50% (Coulter counter).

CC₅₀*: Concentration required to reduce the total cellular DNA content by 50% (DNA hybridization

MTC: Minimal toxic concentration or minimal concentration required to alter normal cell morphology (Visual examination)

CPE: Cytopathic effect assay

We claim:

1. A method of inhibiting viral replication selected from the group consisting of cytomegalovirus (CMV), herpes simplex virus (HSV), influenza, HIV, rhinovirus (RV), Epstein-Barr virus (EBV) and varicella zoster virus (VZV) in a mammal comprising administering to said mammal an anti-viral amount of a compound of formula (I): 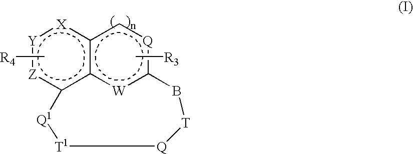
wherein
W is selected from CH, CR₃, CH₂, C═O, CHR₃, N and NR₅; one of X, y, and Z is N or NR₅ while the other two are independently selected from CH, CR₄, CH₂, C═O and CHR₄;
B is selected from the group consisting of: 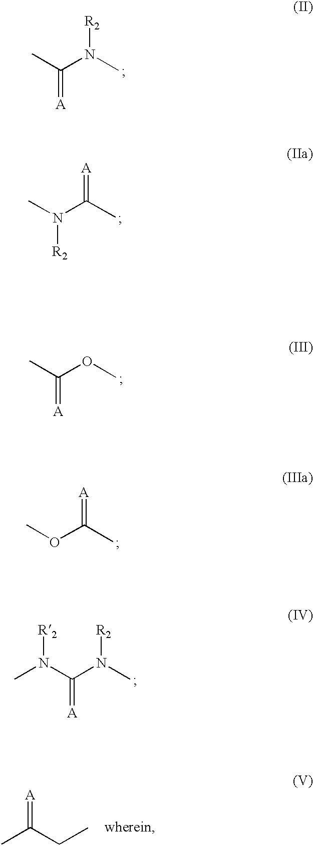
A is O or S;
T and T¹ are independently selected from C₁₋₆ (alkyl, alkoxy, acyl, acyloxy or alkoxycarbonyl), C₂₋₆ alkenyl, C₂₋₆ alkynyl optionally substituted with OH, halogen, amino, mercapto, carboxy or a saturated or unsaturated C₃₋₁₀ (carbocycle or heterocycle) optionally substituted with OH, halogen, amino, mercapto, carboxy, C₁₋₄ (alkyl, alkoxy, alkylthio, acyl, acyloxy or alkoxycarbonyl);
Q and Q¹ are independently selected from N, NR₅, O, S, NH, CH, CHR₃ or a bond;
R₂ and R′₂ are independently selected from H or C₁₋₄ alkyl;
R₃ and R₄ are independently selected from H, OH, halogen, amino, cyano, C₁₋₆ (alkyl, alkoxy, acyl, acyloxy or alkoxycarbonyl), C₂₋₆ alkenyl, C₂₋₆ alkynyl optionally substituted with OH, halogen, amino or C₁₋₄ alkoxy, and saturated or unsaturated C₃₋₁₀ (carbocycle or heterocycle) optionally substituted with OH, halogen, amino, mercapto, C₁₋₄ alkylthio, C₁₋₄ alkoxycarbonyl, halo-substituted C₁₋₄ alkyl or halo-substituted C₁₋₄ alkoxy, C₁₋₄ alkyl, C₁₋₄ alkoxy or C₁₋₄ carboxy;
R₅ is H, C₁₋₆ alkyl or C₁₋₆ acyl optionally substituted with OH, halogen, amino or C₁₋₄ alkoxy; and
n is 0, 1, 2 or 3.

2. A method according to claim 1, wherein W is N or NR₅.

3. A method according to claim 1, wherein Y is N or NR₅ and X and Y are independently selected from CH, CR₄, CH₂, C═O and CHR₄.

4. A method according to claim 1, wherein T is C₁₋₆ alkyl optionally substituted with a saturated or unsaturated C₃₋₁₀ (carbocycle or heterocycle).

5. A method according to claim 1, wherein T′ is C₁₋₆ alkyl optionally substituted with a saturated or unsaturated C₃₋₁₀ (carbocycle or heterocycle).

6. A method according to claim 1, wherein B is

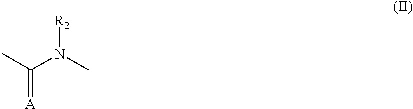

7. A method according to claim 1, wherein B is

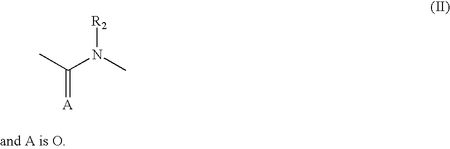

8. A method according to claim 7, wherein T is methyl optionally substituted with a phenyl and Q is O and T′ is allyl and Q¹ is a bond.

9. A method according to claim 7, wherein T is methyl optionally substituted with a phenyl and Q is O and T′ is methyl optionally substituted with a phenyl and Q¹ is a bond.

10. A method according to any one claim 1 to 9, wherein R₃ and R₄ is H and R₂ and R′₂ is H.

11. The method of claim 1 wherein the compound of formula I is

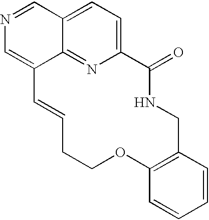

12. The method of claim 1 wherein the compound of formula (I) is

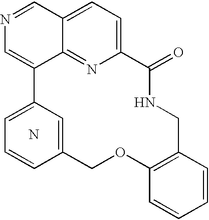

13. The method of claim 1, wherein the compound of formula (I) is

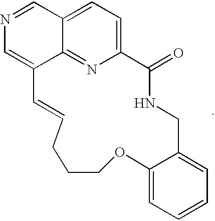

14. The method of claim 1 wherein the viral infection is cytomegalovirus.

15. The method of claim 1 wherein the viral infection is herpes simplex virus.

16. The method of claim 1 wherein the viral infection is influenza.

17. The method of claim 1 wherein the viral infection is selected from the group consisting of HIV, HBV and HCV.

18. The method of claim 1 wherein the viral infection is rhinovirus.

19. The method of claim 1 wherein the viral infection is Epstein-Barr virus.

20. The method of claim 1 wherein the viral infection is varicella zoster virus.

21. A pharmaceutical composition for treating or preventing viral infection selected from the group consisting of cytomegalovirus (CMV), herpes simplex virus (HSV), influenza, HIV, rhinovirus, Epstein-Barr virus (EBV) and varicella zoster virus (VZV) comprising a pharmaceutically acceptable carrier, diluent or adjunct and a compound of formula (I) or a pharmaceutically acceptable salt thereof: 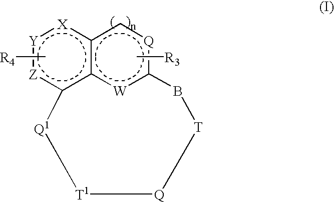
wherein
W is selected from CH, CR₃, CH₂, C═O, CHR₃, N and NR₅; one of X, y, and Z is N or NR₅ while the other two are independently selected from CH, CR₄, CH₂, C═O and CHR₄;
B is selected from the group consisting of: 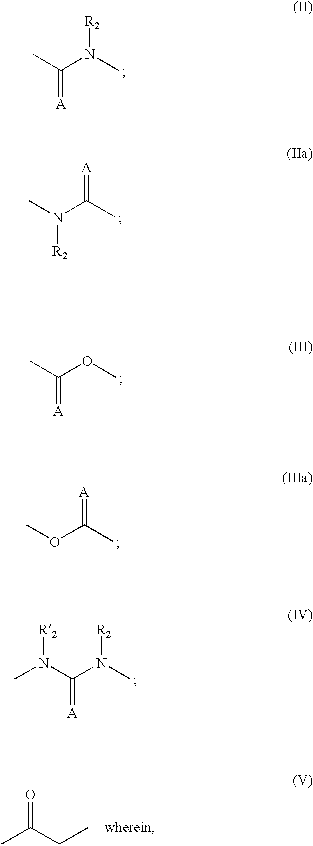
A is O, or S;
T and T¹ are independently selected from C₁₋₆ (alkyl, alkoxy, acyl, acyloxy or alkoxycarbonyl), C₂₋₆ alkenyl, C₂₋₆ alkynyl optionally substituted with OH, halogen, amino, mercapto, carboxy or a saturated or unsaturated C₃₋₁₀ (carbocycle or heterocycle) optionally substituted with OH, halogen, amino, mercapto, carboxy, C₁₋₄ (alkyl, alkoxy, alkylthio, acyl, acyloxy or alkoxycarbonyl);
Q and Q¹ are independently selected from N, NR₅, O, S, NH, CH, CHR₃ or a bond;
R₂ and R′₂ are independently selected from H or C₁₋₄ alkyl;
R₃ and R₄ are independently selected from H, OH, halogen, amino, cyano, C₁₋₆ (alkyl, alkoxy, acyl, acyloxy or alkoxycarbonyl), C₂₋₆ alkenyl, C₂₋₆ alkynyl optionally substituted with OH, halogen, amino or C₁₋₄ alkoxy, and saturated or unsaturated C₃₋₁₀ (carbocycle or heterocycle) optionally substituted with OH, halogen, amino, mercapto, C₁₋₄ alkylthio, C₁₋₄ alkoxycarbonyl, halo-substituted C₁₋₄ alkyl or halo-substituted C₁₋₄ alkoxy, C₁₋₄ alkyl, C₁₋₄ alkoxy or C₁₋₄ carboxy;
R₅ is H, C₁₋₆ alkyl or C₁₋₆ acyl optionally substituted with OH, halogen, amino or C₁₋₄ alkoxy; and n is 0, 1, 2 or 3.

22. A pharmaceutical composition for treating or preventing viral infection selected from the group consisting of cytomegalovirus (CMV), herpes simplex virus (HSV), influenza, HIV, rhinovirus, Epstein-Barr virus (EBV) and varicella zoster virus (VZV) comprising at least one compound as defined in anyone of claims 11, 12 and 13 together with at least one pharmaceutically acceptable carrier or excipient.

23. A compound of formula (I) and pharmaceutical acceptable salts thereof: 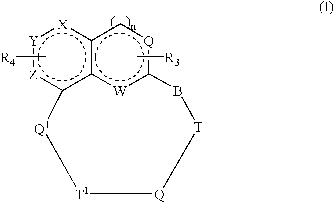
wherein,
B is 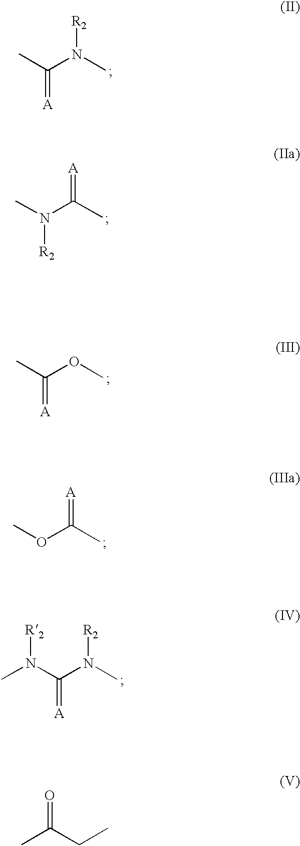
A is O, or S;
T and T¹ are independently selected from C₁₋₆ (alkyl, alkoxy, acyl, acyloxy or alkoxycarbonyl), C₂₋₆ alkenyl, C₂₋₆ alkynyl optionally substituted with OH, halogen, amino, mercapto, carboxy or a saturated or unsaturated C₃₋₁₀ (carbocycle or heterocycle) optionally substituted with OH, halogen, amino, mercapto, carboxy, C₁₋₄ (alkyl, alkoxy, alkylthio, acyl, acyloxy or alkoxycarbonyl);
Q and Q¹ are independently selected from N, NR₅, O, S, NH, CH, CHR₃ or a bond;
R₂ and R′₂ are independently selected from H or C alkyl;
R₃ and R₄ are independently selected from H, OH, halogen, amino, cyano, C₁₋₆ (alkyl, alkoxy, acyl, acyloxy or alkoxycarbonyl), C₂₋₆ alkenyl, C₂₋₆ alkynyl optionally substituted with OH, halogen, amino or C₁₋₄ alkoxy, and saturated or unsaturated C₃₋₁₀ (carbocycle or heterocycle) optionally substituted with OH, halogen, amino, mercapto, C₁₋₄ alkylthio, C₁₋₄ alkoxycarbonyl, halo-substituted C₁₋₄ alkyl or halo-substituted C₁₋₄ alkoxy, C₁₋₄ alkyl, C₁₋₄ alkoxy or C₁₋₄ carboxy;
R₅ is H, C₁₋₆ alkyl or C₁₋₆ acyl optionally substituted with OH, halogen, amino or C₁₋₄ alkoxy; and
n is 0, 1, 2 or 3.

24. A compound according to claim 23, wherein W is N or NR₅.

25. A compound according to claim 23, wherein Y is N or NR₅ and X and Y are independently selected from CH, CR₄, CH₂, C═O and CHR₄.

26. A compound according to claim 23, wherein T is C₁₋₆ alkyl optionally substituted with a saturated or unsaturated C₃₋₁₀ (carbocycle or heterocycle).

27. A compound according to claim 23, wherein T¹ is C₁₋₆ alkyl optionally substituted with a saturated or unsaturated C₃₋₁₀ (carbocycle or heterocycle).

28. A compound according to claim 23, wherein A is O.

29. A compound according to claim 23, wherein A is O and T is methyl optionally substituted with a phenyl and Q is O and T¹ is allyl and Q¹ is a bond.

30. A compound according to claim 23, wherein A is O and T is methyl optionally substituted with a phenyl and Q is O and T¹ is methyl optionally substituted with a phenyl and Q¹ is a bond.

31. A compound according to any one claims 23 to 30, wherein R₃ and R₄ is H and R₂ and R′₂ is H.

32. The compound of claim 23 wherein the compound of formula I is

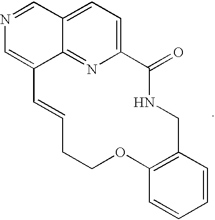

33. The compound of claim 23 wherein the compound of formula is

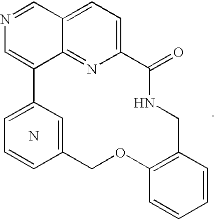

34. The compound of claim 23 wherein the compound of formula is

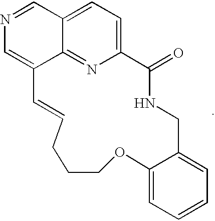

35. The use of a compound according to formula (I) as defined in anyone of claims 23 to 34 for the manufacture of a medicament for treating or preventing a viral infection selected from the group consisting of cytomegalovirus (CMV), herpes simplex virus (HSV), influenza, HIV, rhinovirus, Epstein-Barr virus (EBV) and varicella zoster virus (VZV).
